SEQUENCE LISTING (13 sequences)

SEQ ID NO: 1 - DNA, 2043 bp, Homo sapiens
  CDS (145)..(1443)
ggggagggaggcgggaagagcgcggcacttccgctggccgctggctcgctggccgctcctggaggcggcggcgggagcgcagggggcgcgcggcccggggactcgcattccccggttccccctccaccccacgcggcctggaccatggacgccagatggtgggcagtggtggtgctggctgcgttcccctccctaggggcaggtggggagactcccgaagcccctccggagtcatggacccagctatggttcttccgatttgtggtgaatgctgctggctatgccagctttatggtacctggctacctcctggtgcagtacttcaggcggaagaactacctggagaccggtaggggcctctgctttcccctggtgaaagcttgtgtgtttggcaatgagcccaaggcctctgatgaggttcccctggcgccccgaacagaggcggcagagaccaccccgatgtggcaggccctgaagctgctcttctgtgccacagggctccaggtgtcttatctgacttggggtgtgctgcaggaaagagtgatgacccgcagctatggggccacagccacatcaccgggtgagcgctttacggactcgcagttcctggtgctaatgaaccgagtgctggcactgattgtggctggcctctcctgtgttctctgcaagcagccccggcatggggcacccatgtaccggtactcctttgccagcctgtccaatgtgcttagcagctggtgccaatacgaagctcttaagttcgtcagcttccccacccaggtgctggccaaggcctctaaggtgatccctgtcatgctgatgggaaagcttgtgtctcggcgcagctacgaacactgggagtacctgacagccaccctcatctccattggggtcagcatgtttctgctatccagcggaccagagccccgcagctccccagccaccacactctcaggcctcatcttactggcaggttatattgcttttgacagcttcacctcaaactggcaggatgccctgtttgcctataagatgtcatcggtgcagatgatgtttggggtcaatttcttctcctgcctcttcacagtgggctcactgctagaacagggggccctactggagggaacccgcttcatggggcgacacagtgagtttgctgcccatgccctgctactctccatctgctccgcatgtggccagctcttcatcttttacaccattgggcagtttggggctgccgtcttcaccatcatcatgaccctccgccaggcctttgccatccttctttcctgccttctctatggccacactgtcactgtggtgggagggctgggggtggctgtggtctttgctgccctcctgctcagagtctacgcgcggggccgtctaaagcaacggggaaagaaggctgtgcctgttgagtctcctgtgcagaaggtttgagggtggaaagggcctgaggggtgaagtgaaataggaccctcccaccatccccttctgctgtaacctctgagggagctggctgaaagggcaaaatgcaggtgttttctcagtatcacagaccagctctgcagcaggggattggggagcccaggaggcagccttcccttttgccttaagtcacccatcttccagtaagcagtttattctgagccccgggggtagacagtcctcagtgaggggttttggggagtttggggtcaagagagcataggtaggttccagttactcttcccacaagttcccttaagtcttgccctagctgtgctctgccaccttccagactcactcccctctgcaaatacctgcatttcttaccctggtgagaaaagcacaagcggtgtaggctccaatgctgctttcccaggagggtgaagatggtgctgtgctgaggaaaggggatgcagagccctgcccagcaccaccacctcctatgctcctggatccctaggctctgttccatgagcctgttgcaggttttggtactttagaaatgtaactttttgctcttataattttattttattaaattaaattactgca

SEQ ID NO: 2 - protein, 432 aa, Homo sapiens
MDARWWAVVVLAAFPSLGAGGETPEAPPESWTQLWFFRFVVNAAGYASFMVPGYLLVQYFRRKNYLETGRGLCFPLVKACVFGNEPKASDEVPLAPRTEAAETTPMWQALKLLFCATGLQVSYLTWGVLQERVMTRSYGATATSPGERFTDSQFLVLMNRVLALIVAGLSCVLCKQPRHGAPMYRYSFASLSNVLSSWCQYEALKFVSFPTQVLAKASKVIPVMLMGKLVSRRSYEHWEYLTATLISIGVSMFLLSSGPEPRSSPATTLSGLILLAGYIAFDSFTSNWQDALFAYKMSSVQMMFGVNFFSCLFTVGSLLEQGALLEGTRFMGRHSEFAAHALLLSICSACGQLFIFYTIGQFGAAVFTIIMTLRQAFAILLSCLLYGHTVTVVGGLGVAVVFAALLLRVYARGRLKQRGKKAVPVESPVQKV

SEQ ID NO: 3 - DNA, 31 bp, Artificial Sequence
  5′ primer for 1st PCR
aaaaagcaggcttcgcctggaccatggacgc

SEQ ID NO: 4 - DNA, 31 bp, Artificial Sequence
  3′ primer for 1st PCR
agaaagctgggtcaaccttctgcacaggaga

SEQ ID NO: 5 - DNA, 29 bp, Artificial Sequence
  5′ primer for 2nd PCR
ggggacaagtttgtacaaaaaagcaggct

SEQ ID NO: 6 - DNA, 29 bp, Artificial Sequence
  3′ primer for 2nd PCR
ggggaccactttgtacaagaaagctgggt

SEQ ID NO: 7 - DNA, 30 bp, Artificial Sequence
  5′ primer for PCR
atggacgccagatggtgggcagtggtggtg

SEQ ID NO: 8 - DNA, 30 bp, Artificial Sequence
  3′ primer for PCR
tcaaaccttctgcacaggagactcaacagg

SEQ ID NO: 9 - DNA, 30 bp, Artificial Sequence
  5′ primer for PCR
ggggagggaggcgggaagagcgcggcactt

SEQ ID NO: 10 - DNA, 30 bp, Artificial Sequence
  3′ primer for PCR
tgcagtaatttaatttaataaaataaaatt

SEQ ID NO: 11 - DNA, 16 bp, Artificial Sequence
  5′ primer for RT-PCR
ggcaggccctgaagct

SEQ ID NO: 12 - DNA, 19 bp, Artificial Sequence
  3′ primer for RT-PCR
tgcgggtcatcactctttc

SEQ ID NO: 13 - DNA, 26 bp, Artificial Sequence
  Probe for RT-PCR
ccacagggctccaggtgtcttatctg

(19) United States
(12) Patent Application Publication
(10) Pub. No.: US 2004/0208867 A1
(43) Pub. Date: Oct. 21, 2004

(54) Protein having active sulfate transporter activity and method for detecting canceration of tissue

(75) Inventors: Shoko Nishihara, Bunkyo-ku (JP); Shin Kamiyama, Hachiouji-shi (JP); Hisashi Narimatsu, Tsukuba-shi (JP); Norihiro Kikuchi, Nakano-ku (JP)

Correspondence Address: SUGHRUE MION, PLLC, 2100 PENNSYLVANIA AVENUE, N.W., SUITE 800, WASHINGTON, DC, 20037, US

(73) Assignees: NATIONAL INSTITUTE OF ADVANCED INDUSTRIAL SCIENCE AND TECHNOLOGY; MITSUI KNOWLEDGE INDUSTRY CO., LTD; SEIKAGAKU CORPORATION

(21) Appl. No.: 10/745,662

(22) Filed: Dec. 29, 2003

(30) Foreign Application Priority Data
Dec. 27, 2002 (JP) P. 2002-382123

(51) Int. Cl. G01N 33/574; C07H 21/04; A61K 39/395; C07K 14/705

(52) U.S. Cl. 424/143.1; 435/193; 435/7.23; 530/350; 536/23.5

(57) ABSTRACT

A novel protein having active sulfate transport activity, a nucleic acid en cading such a protein, and a “method for detecting canceration of a tissue” by relating a “detected value of expression in a tissue to be tested” of the above protein to “canceration of the tissue to be tested”.

### BACKGROUND OF THE INVENTION

1. Field of the Invention

The present invention relates to a novel protein having active sulfate transport activity, a nucleic acid encoding the protein, and a “method for detecting canceration of a tissue” by relating a “detected value of expression in a tissue to be tested” of the above protein to “canceration of the tissue to be tested”.

2. Brief Description of the Background Art

As sulk transporter factors, for example, a sulfate ion transporter factor (Cell, 78(6), 1073-1087 (1994)), active sulfate transporter factors (Proc. Natl. Acad. Sci. USA, 91, 10707-10711 (1994) (active sulfate transporter actor of 75 kDa in molecular weight) and Biochemistry, 35, 3695-3703 (1996) (active sulfate transporter factor of 230 kDa in molecular weight)) and the like have been known, but genetic engineering techniques for mass synthesis of active sulfate transporter factors through cloning have not been established.

On the other hand, various method are known as the method for detecting canceration of tissues, and examples include X-ray inspection, endoscopy and inspection of tumor markers such as CA-19-9. Definite diagnosis cannot be made by X-ray inspection, endoscopy and the like, because tissues can be observed only from the outside, and tumor markers are also insufficient for definite diagnosis from the viewpoint of generating false positive and false negative.

Definite diagnosis of canceration of a tissue is carded out in reality by a method in which the tissue is collected by biopsy and the tissue is confirmed by carrying out its culturing, but this method requires a certain period of time for the tissue culturing.

On the other hand, a surgical operation in which a lesion part of a tissue of the living body is excised by a surgical method under an endoscope is generally carried out. For example, in case that there is a technique for conveniently verifying the presence or absence of canceration on such a lesion part, it can be led to the early detection of cancellation and also can be used for the subsequent treatment and prevention thereof of the patient.

In addition, JP-A-11-157190 discloses that detection of gastric cancer and pancreatic cancer can be carried out by detecting a DNA encoding N-acetylglucosamine transferase and relating its “change in expression” to the “gastric cancer or pancreatic cancer”.

Since discovery of a new protein having active sulfate transport activity leads to the elucidation of the mechanism of material transportation in the living body, great concern has been directed toward such a discovery. Great concern has also been directed toward the development of “a method for detecting canceration of a tissue” from the collected tissue as soon as possible with a high reliability.

### SUMMARY OF THE INVENTION

In order to solve the above problems the present inventors have conducted intensive studies and found a novel nucleic acid as a result and discovered thereafter that a protein encoded by the nucleic acid has a new “active sulfate transport activity” and that the “expressed amount of the protein in a tissue of canceration” is increased in comparison with the “expressed amount in a healthy tissue”, and the present invention has been accomplished by applying this to a detection method of canceration of tissues

The present invention relates to the following (1) to (14):

(1) A protein of the following (a) or (b):

(a) a protein comprising the amino acid sequence represented by SEQ ID NO:2,

(b) a protein comprising an amino acid, sequence in which 1 to 21 amino acid residue(s) are substituted, deleted, inserted or transposed in the amino acid sequence represented by SEQ ID NO:2 and also having active sulfate transport activity.

(2). The protein according to (1) wherein the active sulfate transport activity is 3′-phosphoadenosine5′-phosphosulfate transport activity.

(3) An active sulfate transporter agent which comprises the protein according to (1) or (2) as an active ingredient.

(4) A method for transporting active sulfate which comprises contacting active sulfate with the protein according to (1) or (2).

(5) A method for detecting canceration of a tissue to be tested which comprises relating a detected value of expression in a tissue to be tested of the protein according to (1) or (2) to canceration of the tissue to be tested.

(6) The method according to (5), wherein the detected value of expression in a tissue to be tested of the protein according to (1) or (2) is a difference obtained by determining an expressed amount of a nucleic acid encoding the protein according to (1) or (2) and comparing the value obtained by the determination with the expressed amount of the nucleic acid in a healthy tissue.

(7) The method according to (6), wherein the expressed amount of a nucleic acid encoding the protein according to (1) or (2) is the expressed amount of a nucleic acid comprising the nucleotide sequence of the following (A) or (B):

(A) a nucleotide sequence of 30 to 1,500 bp comprising a part of the nucleotide sequence represented by SEQ ID NO:1,

(B) a nucleotide sequence of 30 to 1,500 bp comprising a nucleotide sequence complementary to a part of the nucleotide sequence represented by SEQ ID NO: 1.

(8) The method according to (7), wherein the part of the nucleotide sequence of SEQ ID NO:1 is a nucleotide sequence of nucleotide numbers 145 to 1,443 represented by SEQ ID NO:1 or a nucleotide sequence of nucleotide numbers 464 to 553 represented by SEQ ID NO:1.

(9) The method according to any one of (5) to (8), wherein the tissue to be tested is a tissue derived from the stomach or the large intestine.

(10) A nucleic acid of 30 to 1,500 bp which comprises a part of the full nucleotide sequence represented by SEQ ID NO:1, or a nucleotide sequence complementary to the part of the nucleotide sequence.

11) The nucleic acid according to (10), which comprises a nucleotide sequence of nucleotide number 464 to 553 represented by SEQ ID NO:1, or a nucleotide sequence complementary to the nucleotide sequence.

(12) The nucleic acid according to (10), which comprises a nucleotide sequence of nucleotide numbers 145 to 1,443 represented by SEQ ID NO:1, or a nucleotide sequence complementary to the nucleotide sequence.

(13) The nucleic acid according to (10), which consists of a nucleotide sequence of nucleotide numbers 145 to 1,443 represented by SEQ ID NO:1, or a nucleotide sequence complementary to the nucleotide sequence.

(14) The nucleic acid according to any one of (10) to (13), which is a DNA.

### BRIEF DESCRIPTION OF THE DRAWINGS

FIG. 1 shows fractions in which the PAPS transport activity of the protein of the present invention is present. Each white bar indicates a negative control.

FIG. 2 shows transport activity of the protein of the present invention for UDP-GlcNAc, GDP-Fuc, UDP Gal, CMP-Sia, UDP-Glc, UDP-GalNAc, UDP-GlcA, GDP-Man and PAPS. Each white bar indicates a negative control.

FIG. 3 shows influence of PAPS concentration on the PAPS transport activity of the protein of the present invention.

FIG. 4 shows a Km value of the protein of the present invention.

FIG. 5 shows expressed amounts of STP3 gene transcript in canceration-caused tissues and healthy tissues of tissues of the stomach. Each white bar indicates an expressed amount of STP3 gene transcript in a healthy tissue (relative value to the expressed amount of β-actin gene transcript), and each black bar indicates an expressed amount of STP3 gene transcript in a canceration-caused tissue (relative value to the expressed amount of β-actin gene transcript).

FIG. 6 shows expressed amounts of STP3 gene transcript in canceration-caused tissues and healthy tissues of tissues of the large intestine. Each white bar indicates an expressed amount of STP3 gene transcript in a healthy tissue (relative value to the expressed amount of β-actin gene transcript), and each black bar indicates an expressed amount of STP3 gene transcript in a canceration-caused tissue (relative value to the expressed amount of β-actin gene transcript).

FIG. 7 shows detected amounts of CA19-9 in canceration-caused tissues of the large intestine.

### DETAILED DESCRIPTION OF THE INVENTION

The present invention is explained below in detail based on the embodiments of the present invention.

### (1) Protein of the Present Invention

The protein of the present invention is a protein of the following (a) or (b):

(a) a protein comprising the amino acid sequence represented by SEQ ID NO:2,

(b) a protein comprising an amino acid sequence in which 1 to 21 amino acid residue(s) are substituted, deleted, inserted or transposed in the amino acid sequence represented by SEQ ID NO:2 and also having active sulfate transport activity

Among the proteins of the present invention, the protein shown by the above (a) comprises a protein encoded by a region of nucleotide numbers 145 to 1,443 (coding region: CDS) in the nucleotide sequence represented by SEQ ID NO:1.

It is known in general that the enzyme activity of a protein having enzyme activity is maintained even when one or two or more of the constituting amino acids of its amino acid sequence are substituted, deleted, inserted or transposed, and a protein having such a mutation is a variant of the same protein. Also in the case of the above protein (a) among the proteins of the preset invention, when one or two or more (from 2 to 21) of the constituting amino acid residues of its amino acid sequence represented by SEQ ID NO:2 are substituted, deleted, inserted or transposed, the substance is a substance substantially identical to the above protein (a), so long as that it keeps transport activity to transport an active sulfate, so that it is included in the proteins of the present invention The protein having such a mutation is a protein encoded by a nucleotide sequence having substitution, deletion, insertion or transposition of one or two or more (preferably from 2 to 63) nucleotides in a region of nucleotide numbers 145 to 1,443 represented by SEQ ID NO:1.

It is preferable that the amino acid sequence of a protein having such a mutation has 95% or more, preferably 96% or more, and more preferably 97% or more, of homology with the amino acid sequence of the above protein (a). Homology of amino acid sequences can be easily calculated using commonly known computer software such as FASTA, and the use of such a software can also be provided from internet.

That is, mutation of amino acids can such as substitution, deletion, insertion or transposition occurs in the amino acid sequences of naturally existing proteins due to polymorphism and mutation of the gene DNA encoding the same and also by the modification reaction and the like of the produced proteins in the cells and during the production, but in spite of this, it is known that some of them show physiological and biological activities which are substantially identical to those of the original proteins having no mutation. Thus, a protein having a substantially slight difference but no great difference in terms of its function is also included in the above “protein”. A case in which the above mutation is artificially introduced into the amino acid sequence of a protein is the same, and in this case, it is possible to prepare various “protein having mutations”. For example, it is known that a protein in which a certain cysteine residue in the amino acid sequence of human interleukin 2 (IL-2) is substituted with a serine residue keeps the IL-2 activity (Science, 224,1431 (1984)). It is also known that a certain protein has a peptide region which is not essential for the activity. For example, a signal peptide existing in a protein to be secreted into the extracellular moiety, and a pro-sequence which is found in a protease precursor or the like correspond to this case, and most of these regions are removed after their translation or while converting into active proteins. Although a protein having a sequence of such a peptide region which is not essential for the activity is present in the form of different secondary structure, it is a protein having almost the same function, and such a sequence may be connected to the “protein of the present invention”. Such a “protein having mutation” can be easily prepared by “site-directed mutagenesis” and the like conventionally known methods.

Regarding the active sulfate to be transported by the “protein of the present invention”, adenosine 5′-phosphosulfate (hereinafter referred sometimes to as “APS”) and 3′-phosphoadenosine 5′-phosphosulfate (hereinafter sometimes referred to as “PAPS”) are preferred, and PAPS is particularly preferred.

Confirmation of the “active sulfate transport activity” possessed by the “protein of the present invention” can be carried out, or example, in accordance with the method of J. Biol. Chem., 275, 13580-13587 (2000). That is, it can be carried out by a method in which an “active sulfate (PAPS, etc.)” labeled with a radioisotope such as [¹⁴C], [³H] or [³⁵P] ([³⁵P] is preferred) is mixed with the above “protein of the present invention”, a yeast membrane fraction is added thereto, and the isotope transferred to the membrane fraction is measured. The method includes a method which will be described later in Example 2.

### (2) Nucleic Acid of the Present Invention

The “nucleic acid of the present invention” is a nucleic acid of 30 to 1,500 bp comprising a part of the full nucleotide sequence represented by SEQ ID NO:1 or a nucleotide sequence complementary to the part of the nucleotide sequence.

The “part of the full nucleotide sequence represented by SEQ ID NO:1” according to the “nucleic acid of the present invention” comprises a length of 30 to 1,500 bp, preferably 40 to 1,450 bp, more preferably 60 to 1,400 bp, and most preferably 80 to 1,300 bp. Examples of the nucleic acid include a nucleic acid comprising a nucleotide sequence of nucleotide numbers 464 to 553 represented by SEQ ID NO:1 and a nucleic acid comprising a nucleotide sequence of nucleotide numbers 145 to 1,443 represented by SEQ ID NO:1. The nucleic acid or the nucleic acid comprising a nucleotide sequence complementary to the same can be used in the “detection method of the present invention” which will be described later, and since the nucleic acid having such a length particularly has both hybridizing ability and easy handling, it is excellent as a probe for hybridization. Even in the case of a nucleic acid other than the nucleic acids exemplified in the above, it can be optionally selected from the nucleotide sequence represented by SEQ ID NO:1 within the range of length exemplified in the above, particularly within the range of nucleotide numbers 145 to 1,443.

Also, the unit “bp” as used herein representing the length of a nucleic acid is a length of a nucleic acid converted to a number corresponding to the number of base pairs which form a double-stranded structure when nucleic acids form a double strand, or in the case of a single-stranded nucleic acid, to the number of base pairs of a double-stranded structure in which a single strand comprising a nucleotide sequence complementary to the nucleic acid is hybridized. Accordingly, for example, a single-stranded DNA of “1,000 bp” is formed from 1,000 nucleotides, and a double-stranded DNA of “1,000 bp” is formed from 2,000 nucleotides (1,000 pairs of nucleotides), and both of these cases represent the same DNA of “a chain length comprising 1,000 nucleotides”

In addition, the nucleic acid comprising the nucleotide sequence represented by SEQ ID NO:1 or the nucleic acid comprising a nucleotide sequence comprising nucleotide numbers 145 to 1,443 of the nucleotide sequence contains a nucleotide sequence which corresponds to a termination codon in the protein synthesis, so that it is useful because it can be used for preparing a “polypeptide comprising an amino acid sequence of amino acid numbers 1 to 432 represented by SEQ ID NO:2” by genetic engineering techniques.

The “nucleic acid of the present invention” may be either a DNA or an RNA, but it is preferably a DNA which is excellent in terms of the stability when used as a probe for hybridization or used for the preparation of a recombinant vector or a recombinant in the “Detection method of the present invention” which will be described later.

In addition, a nucleic acid (particularly a DNA) which hybridizes with the above “nucleic acid having a part of the nucleotide sequence represented by SEQ ID NO:1 or a nucleic acid comprising a nucleotide sequence complementary thereto” under stringent conditions can also be used as a probe for hybridization, for example for inspecting expressing conditions of a nucleic acid having the nucleotide sequence represented by SEQ ID NO:1 in the living body, and it is markedly useful as a reagent or a diagnostic drug for studies on medical science, biochemistry and the like.

Also, the term “under stringent conditions” as used herein means conditions generally used in the “experimental techniques which use hybridization of nucleic acid (e.g. Northern blot hybridization and Southern blot hybridization)” and the like, and its preferred example is conditions at 42° C. in the presence of 37.5% formamide, 5×SSPE (sodium chloride/sodium phosphate/EDTA (ethylenediaminetetraacetic acid) buffer solution), 5×Denhardt's solution and 0.5% SDS (sodium dodecyl sulfate)

It is possible to prepare the “nucleic acid of the present invention”, for example, by the following method.

A clone containing total sequence of SEQ ID NO:1 (GenBank accession No. XM_{—}059770) can be obtained by carrying out retrieval of nucleotide sequences by BLAST using the nucleotide sequence (GenBank accession No. NM_{—}005827) of a known UDP-galactose transporter-related gene (human UDP-galactose transporter related: UGTREL 1) as the query. Based on its complementary sequence, the nucleic acid of the present invention (e.g., the DNA of the present invention) can be prepared by amplifying it by conventionally known method such as polymerase chain reaction (hereinafter sometimes referred to as “PCR”) from a cDNA library or the like using PCR or the like.

The “nucleic acid of the present invention” can be prepared by carrying out a primary PCR using the sequence represented by SEQ ID NO:3 as a 5′ primer, and the sequence represented by SEQ ID NO:4 as a 3′ primer, in accordance with a usual method using, for example, a human cDNA library as the template, and further carrying out a secondary PCR using the sequence represented by SEQ ID NO:5 as a 5′ primer, and the sequence represented by SEQ ID NO:6 as a 3′ primer and using the above primary PCR product as the template.

In the nucleotide sequence represented by SEQ ID NO:1, a region considered to encode the polypeptide (nucleotide numbers 145 to 1,443) can be prepared by PCR using a primer comprising the nucleotide sequence represented by SEQ ID NO:7 as a 5′ primer, and a primer comprising the nucleotide sequence represented by SEQ ID NO:8 as a 3′ primer, and the nucleic acid comprising the full nucleotide sequence represented by SEQ ID NO:1 can be prepared by PCR, for example, using the nucleotide sequence represented by SEQ ID NO:9 as a 5′ primer, and the nucleotide sequence represented by SEQ ID NO:10 as a 3′ primer, and using a commercially available cDNA library as the template. Also, a region comprising a nucleotide sequence of nucleotide numbers 464 to 553 of SEQ ID NO:1 can be prepared in the same manner from a cDNA library using a prime comprising the nucleotide sequence of SEQ ID NO:11 as a 5′ primer, and a primer comprising the nucleotide sequence of SEQ ID NO:12 as a 3′ primer.

In this case a DNA fragment of 1,332 bp is obtained as the product of the PCR using the primers of SEQ ID NO:3 and SEQ ID NO:4, a DNA fragment of 1,366 bp is obtained as the product of the PCR using the primers of SEQ ID NO:5 and SEQ ID NO:6, and a DNA fragment of about 1.3 kbp is obtained as the product of the PCR which used the primers of SEQ ID NO:7 and SEQ ID NO:8. The “nucleic acid of the present invention” can be obtained by separating each of these products by a method which screens DNA fragments based on their molecular weights such as agarose gel electrophoresis, and then isolating it in accordance with a usual method such as a method which cuts out a specified band.

The thus isolated “nucleic acid of the present invention” can be used for preparing a recombinant which expresses the “polypeptide” encoded thereby. That is, by connecting a restriction end (cohesive end or blunt end) to both termini of the “nucleic acid of the present invention” by a usual method, it can be inserted into an expression vector. Those skilled in the art can optionally select a restriction end suited for the expression vector. Those skilled in the art can optionally select an expression vector suitable for the “host cell capable of expressing a protein encoded by the nucleic acid of the present invention”. It is preferable that regions relating to the gene expression (a promoter region, an enhancer region, an operator region and the like) are appropriately arranged in such an expression vector so that the above “nucleic acid of the present invention” can be expressed in the desired host cell, and that the vector is constructed in such a manner that the “nucleic acid of the present invention” can be appropriately expressed.

A recombinant can be obtained by integrating the above “expression vector containing the nucleic acid of the present invention” into a host cell. As the above “host cell”, either a eucaryotic cell (mammalian cell, yeast, insect cell or the like) or a procaryotic cell (Escherichia coli, Bacillus subtilis or the like) can be used. When a eucaryotic cell is used as the host cell an “expression vector for eucaryotic cell” is selected as a vector which is used as the basis of the “expression vector containing the nucleic acid of the present invention” (hereinafter sometimes referred to as “basic vector”), and when a procaryotic cell is used as the host cell, an “expression vector for procaryotic cell” is selected as the basic vector.

Also, since the “nucleic acid of the present invention” is a nucleic acid discovered from a human genomic library and considered to be a gene relating to a transporter factor, it is considered that, in the present invention, a “protein of the present invention” having properties more closer to those of a natural one (e.g., a protein to which a sugar chain is added) can be obtained when an eucaryotic cell is used as the host cell of the recombinant. Accordingly, it is preferable to select an eucaryotic cell, preferably a mammalian cell or a yeast, particularly a mammalian cell, as the “host cell”, and it is preferable to select a vector for eucaryotic cell, particularly a vector for mammalian cell, as the basic vector of the “expression vector containing the nucleic acid of the present invention”.

In recent years, techniques in which a transformant is cultured or grown and a substance of interest is isolated and purified from its culture mixture or grown product have been established as genetic engineering techniques. It is preferable that the “expression vector containing the nucleic acid of the present invention” is constructed in such a manner that the “polypeptide” encoded by its “nucleic acid of the present invention ” can be easily isolated and purified. Particularly, since isolation and purification can be easily carried out, it is preferable to prepare “polypeptide” according to genetic engineering techniques by constructing an “expression vector containing the nucleic acid of the present invention” in such a manner that the above “polypeptide” is expressed as a form of a “fusion protein” with a “label peptide”.

An example of the above “label peptide” is a peptide having a function to facilitate secretion, separation, purification or detection of a “polypeptide encoded by the nucleic acid of the present invention” from the grown product of a transformant, by =expressing the “polypeptide” as a “fusion protein” bound with a “label peptide” in preparing the “polypeptide encoded by the nucleic acid of the present invention” by genetic recombination techniques. Examples of the “label peptide” include peptides such as a signal peptide (a peptide comprising 15 to 30 amino acid residues, which is present on the N-terminus of many proteins and performs a function in cells for the selection of protein in the intracellular membrane permeation mechanism; e.g., OmpA, OmpT, Dsb, etc.), protein kinase A, protein A (protein of about 42,000 in molecular weight, which is a constituting component of Staphylococcus aureus cell wall), glutathione S transferase, His tag (sequence in which 6 to 10 hisitidine residues are arranged), myc tag (13 amino acid sequence derived from cMyc protein), FLAG peptide (analysis marker comprising 8 amino acid residues), T7 tag (comprising the first 11 amino acid residues of gene 10 protein), S tag (comprising 15 amino acid residues, derived from pancreas RNase A), HSV tag, pelB (22 amino acid sequence of E coli outer membrane protein pelB), HA tag (comprising hemagglutinin origin 10 amino acid residues), Trx tag (thioredoxin sequence), CBP tag (calmodulin binding peptide), CBD tag (cellulose binding domain), CBR tag (collagen binding domain), β-lac/blu (β-lactamase), β-gal (β-galactosidase), luc (luciferase), HP-Thio (His-patch thioredoxin), HSP (heat shock peptide), Lnγ (laminin γ peptide), Fn (fibronectin partial peptide), GFP (green fluorescence peptide), YFP (yellow fluorescnce peptide), CFP (cyan fluorescence peptide), BFP (blue fluorescence peptide), DsRed and DsRed2 (red fluorescence peptides), MBP (maltose binding peptide), LacZ (lactose operator), IgG (immunoglobulin G), and avidin, protein G, and any one of these label peptides can be used. Among these, the signal peptide, protein kinase A, protein A, glutathione S transferase, His tag, myc tag, FLAG peptide, T7 tags S tag, HSV tag, pelB or HA tag is particularly preferable, because expression of the protein of the present invention by genetic engineering techniques and its purification become more easy. Particularly, it is a fusion protein with HA tag, it is preferable because expression of the “polypeptide encoded by the nucleic acid of the present invention” can be confirmed easily.

Examples of the basic vector which can be expressed in mammalian cells and can produce the above “polypeptide encoded by the nucleic acid of the present invention” as a fusion protein with HA tag include YEP352 GAPII or pYES-DEST52 (manufactured by Invitrogen) and the like, though those skilled in the art can select an appropriate basic vector by taking into consideration the host cell, restriction enzymes, marker peptide and the like to be used in the expression of the “polypeptide encoded by the nucleic acid of the present invention”.

Also, since the “nucleotide sequence of the nucleic acid of the present invention” has been disclosed by the present invention, those skilled in the art can easily prepare a region of interest by amplifying it by a method such as PCR using primers optionally prepared based on the nucleotide sequences of both termini of the “nucleic acid of the present invention” of interest or of a “partial region of the nucleic acid of the present invention” to be prepared.

### (3) Detection Method of the Present Invention

The “detection method of the present invention” is a method for the detection of canceration of a tissue to be tested, which comprises relating “detected value of expression in a tissue to be tested” of the protein of the present invention to “canceration of the tissue to be tested”.

Detection of the expressed amount of the protein of the present invention in the “detection method of the present invention” can be carried out for example, by measuring the active sulfate transport activity possessed by the protein of the present invention, and it can also be detected by measuring the expressed amount of the above nucleic acid of the present invention which can be easily carried out, for example, by measuring changes in the expressed amount of the “DNA containing nucleotide numbers 464 to 553 represented by SEQ ED NO:1” as a nucleic acid of the present invention.

Expressed amount of the above DNA can be determined by using for example, quantitative real time PCR (hereinafter sometimes referred to as “quantitative RT-PCR”) using primers prepared based on the 5′-end and 3′-end nucleotide sequences of the DNA exemplified in the above and a probe prepared by linking a fluorescence dye to a quenching matter. The probe used in the RT-PCR includes, for example, a nucleic acid comprising the nucleotide sequence represented by SEQ ID NO:13.

As the tissue to be used in the “detection method of the present invention”, any tissue can be used, but the gullet, the stomach, the lungs, the pancreas, the liver, the kidney, the duodenum, the small intestines, the large intestine, the rectum, the colon and the like can be exemplified, of which the gullet, the stomach, the small intestines and the large intestine are preferably exemplified and the stomach and the large intestine are most preferably exemplified.

It is preferable to carry out the “detection method of the present invention” using a tissue slicer obtained by separating from a tissue. That is, since the expressed amount can be detected by comparing expressed amount of the above DNA in a lesion moiety (a tissue to be tested) contained in a tissue obtained by a biopsy or the like with expressed amount of the above DNA in a healthy part in the periphery of the moiety, it is usefull in the diagnosis of cancer, progress observation in cancer treatment and the like.

In addition, although “determination of the expressed amount of DNA” in the “detection method of the present invention” can be carried out by a method such as PCR which measures the amount of a “DNA” or “mRNA formed by its transcription from the DNA” by amplifying it, but it is not necessarily limited to the determination of a “DNA” or “mRNA formed by its transcription from the DNA”, and it is possible to measure the amount by determining, for example, the “polypeptide encoded by the nucleic acid of the present invention” formed through the transcription and translation by the above DNA, Such a “determination of polypeptide” can be carried out in accordance with a conventional method (Western blotting enzyme immunoassay or the like) using, for example, an antibody prepared in the usual way using the purified “polypeptide encoded by the nucleic acid of the present invention”. Among these methods, the PCR is particularly preferable, and RT-PCR is most preferable.

Regarding the canceration of a tissue, it is preferable to judge that a tissue to be tested is causing canceration, preferably when expressed amount of the above DNA in the tissue to be tested is increasing in comparison with a healthy tissue, and when the canceration is detected particularly by the RT-PCR, it can be judged that a tissue to be tested is causing canceration when expressed amount of the above DNA in the tissue to be tested is increasing by a factor of 3% or more, preferably 5% or more, most preferably 10% or more, in comparison with a healthy tissue.

The protein and the nucleic acid of the present invention can be used as the active sulfate transporter agent or in the method for detect canceration of a tissue to be tested, together with a carrier or a diluent.

The present invention is further described below in detail based on Examples.

### EXAMPLE 1

### Preparation of the Nucleic Acid of the Present Invention

BLAST retrieval was carried out using the nucleotide sequence (GenBank accession No. MN_{—}005827) of a human UDP-galactose transporter-related gene (UGTREL 1) as the query. As a result, it was found that the nucleotide sequence of STP3 (GenBank accession No. XM_{—}059770) as shown in SEQ ID NO:1 has homology. The amino acid sequence encode by this nucleotide sequence was deduced as SEQ ID NO:2.

In order to obtain a DNA comprising the nucleotide sequence of SEQ ID NO:1, using a human large intestine-derived cDNA library (manufactured by Clontech) as the template, primary PCR was carried out using DNA fragments comprising the sequence represented by SEQ ID NO:3 as a 5′ primer, and the sequence represented by SEQ ID NO:4 as a 3′ primer, in accordance with a usual method, and then secondary PCR was carried out using the nucleotide sequence represented by SEQ ID NO:5 as a 5′ primer, and the nucleotide sequence represented by SEQ ID NO:6 as a 3′ primer. A DNA fragment of about 1.37 kbp was recovered from the PCR products in accordance with a conventional method using agarose gel electrophoresis.

### EXAMPLE 2

A recombinant vector YEP352 GAPII-STP3 was obtained by inserting the DNA fragment obtained in Example 1 into a yeast expression vector YEP352 GAPII in accordance with the instructions of the Gateway Cloning System (manufactured by Invitrogen). Transformants were obtained in accordance with the usual way by transferring this vector into a yeast strain W303a which requires uracil for its growth. A transformant was selected by culturing the transformants obtained in this manner on an agar plate containing a uracil-free SD medium, and the thus obtained transformant was mass-cultured using a uracil-free SD liquid medium.

Cells of the thus culture transformant were washed with distilled water containing 10 mmol/l NaN₃, and then about 5 g of the transformant cells were suspended in 5 volumes of a lysis solution (a solution of pH 7.5 containing 1.4 mol/l sorbitol, 50 mmol/l potassium phosphate, 10 mmol/l NaN₃ and 40 mmol/l 2-mercaptoethanol), mixed with Zymolyase 100T (manufactured by Seikagaku Corporation) to a concentration of 1 mg/g cells, and then allowed to undergo the reaction at 37° C. for 30 minutes to effect lysis of the cell wall of the transformant, Thereafter the resulting transformant cells were washed twice with 0.8 mol/l of a sorbitol solution, suspended in 10 ml of a lysis solution (a solution of pH 7.2 containing 0.8 mol/l sorbitol, 10 mmol/l triethanolamine, pepstain A and 1 mmol/l phenylmethylsulfonyl fluoride), and then homogenized using Downs homogenizer. This was centrifuged at 1,000×g for 10 minutes at 4° C., and the supernatant fluid was recovered and used as a cell extract.

This cell tact was firstly subjected to 15 minutes of ultracentrifugation at 10,000×g and at 4° C., and the precipitate was recovered. The fraction comprising this precipitate (P10) was a fraction rich in endoplasmic reticulum. The supernatant fluid after removal of the P10 was then subjected to 1 hour of ultracentrifugation at 100,000×g and at 4° C., and the precipitate was recovered. The fraction comprising this precipitate (P100) was a fraction rich in the Golgi body.

The ultracentrifugation supernatant fluid after removal of the P100 was named S100 and used as a cytosol fraction.

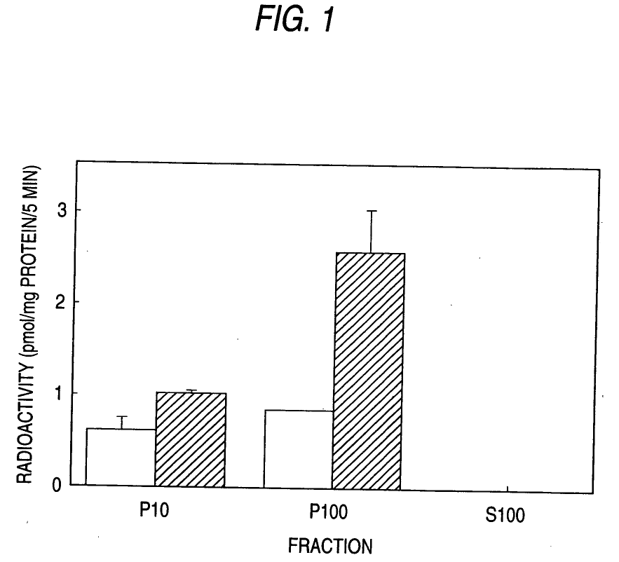

The reaction was started by adding 200 μg protein of P10, P100 or S100 fraction to 100 μl of a reaction solution (20 mM Tris-HCl buffer (pH 7.5) containing 0.25 M sucrose, 5 mM MgCl₂, 1 mM MnCl₂, 10 mM 2-mercaptoethanol and 1 μM ³⁵S-labeled PAPS). Five minutes thereafter, 10 volumes of an ice-cooled reaction terminating solution (20 mM Tris-HCl buffer (pH 7.5) containing 0.25 M sucrose, 5 mM MgCl₂ and 150 mM KCl) was added thereto, and the mixture was filtered through a nitrocellulose filter of 0.45 μm in pore size. This filter was washed with 10 ml of the iced-cooled reaction terminating solution, and the radioactivity of this filter was measured using a liquid scintillation counter (FIG. 1). Respective fractions prepared using a transformant of a yeast strain W303a transformed with YER352 GAPII alone were used as negative controls.

As a result strong PAPS transport activity was observed in the P100 fraction.

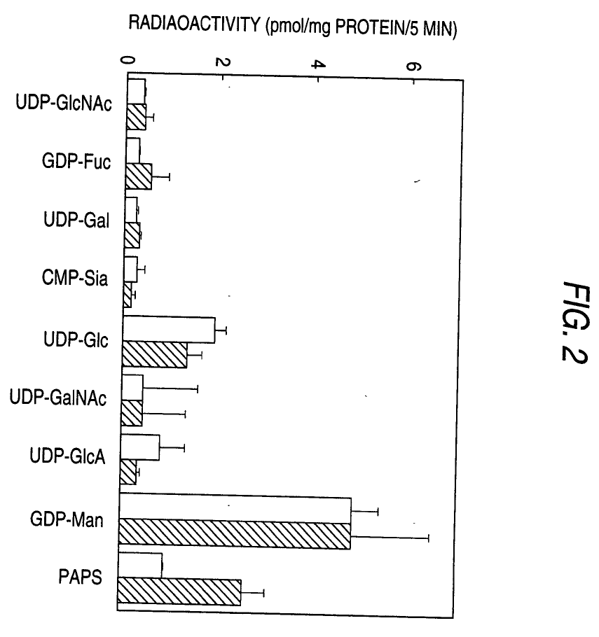

Thereafter, the transport activity of the P100 fraction was measured using ³H-labeled uridine diphosphate-N-acetiglucosamine (UDP-GlcNAc), ³H-labeled guanosine diphosphate-fucose (GDP-Fuc), ³H-labeled uridine diphosphate-galactose (UDP-Gal), ³H-labeled cytidine monophosphate-sialic acid (CMP-Sia), ³H-labeled uridine diphosphate-glucose (UDP-Glc), ³H-labeled uridine diphosphate-N-acetylgalactosamine (UDP-GalNAc), ¹⁴C-labeled uridine diphosphate-glucuronic acid (UDP-GlcA) and ³H-labeled guanosine diphosphate-mannose (GDP-Man), instead of the ³⁵S-labeled PAPS (FIG. 2).

As, a result, it was found that the protein of the present invention substantially has no activity to specifically transport substances other than PAPS.

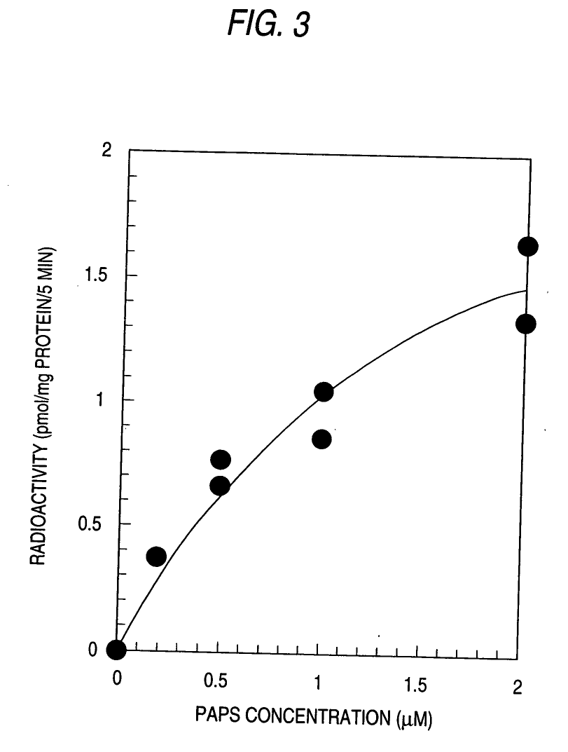

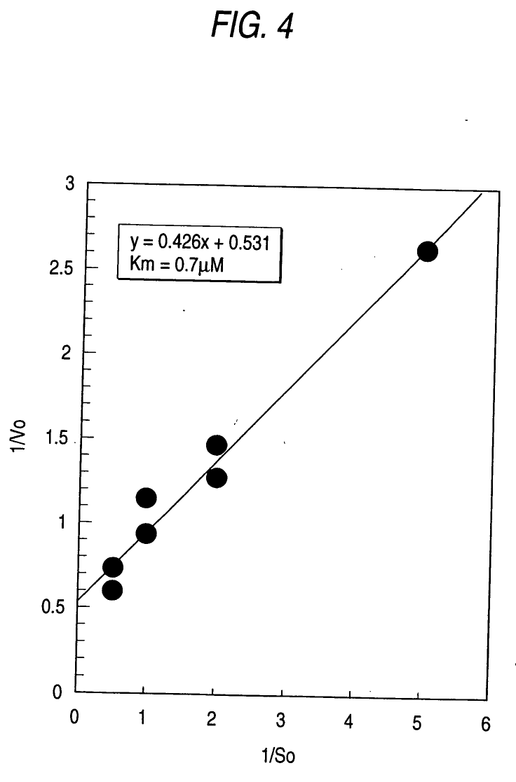

In addition, when change in the transport activity was measured by changing concentration of PAPS to be added (FIG. 3), and Km value (μM) of the protein of the present invention was calculated from the measured values it was found that the Km value was 0.7 μM (FIG. 4).

### EXAMPLE 3

### Change in the Expressed Amount of the DNA of the Present Invention in Gastric Cancer Tissue

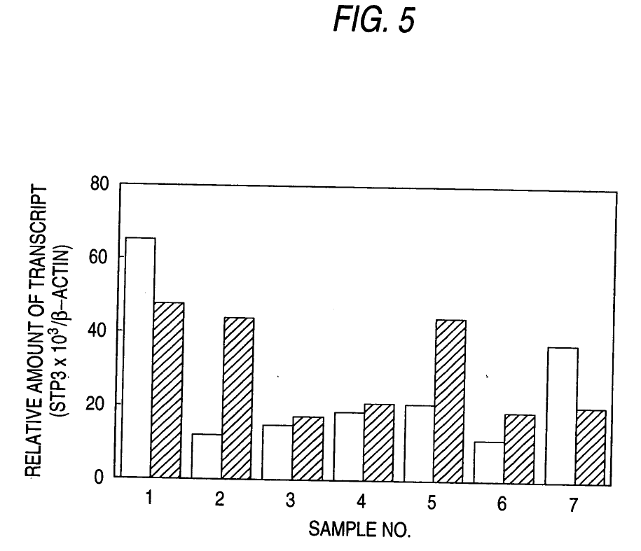

Using quantitative real time PCR, the expressed amount of the DNA of the present invention in a human gastric cancer tissue was compared with that of a healthy stomach tissue of the same patient. RNA was extracted from a human gastric cancer tissue or a healthy stomach tissue using RNease Mini Kit (manufactured by Qiagen) and converted into a single strand DNA by a oligo(dT) method using Super-Script First-strand Synthesis System (manufactured by Invitrogen). Using this DNA as the template, a quantitative real time PCR was carried out by ABI PRISM 7700 (manufactured by Applied Biosystems) using primes (5′ primer; SEQ ID NO:11, 3′ primer: SEQ ID NO:12) and a TaqMan probe (SEQ ID NO:13). The PCR was carried out by heating at 95° C. for 10 minutes and then 40 cycles of a reaction at 95° C. for 15 seconds and at 60° C. for 1 minute as one cycle. Using a β-actin gene as an internal standard, comparison was made by calculating ratio of the expressed amount of the gene transcript to the expressed amount of the β-actin gene (b-Act) transcript (Table 1, FIG. 5).

TABLE 1

| | Healthy tissue | | Canceration tissue | | Canceration |
|---|---|---|---|---|---|
| Sample | Measured | STP3/ | Measured | | tissue/healthy |
| No. | value | b-Act | value | STP3/b-Act | tissue |
| 1 | 14093.05 | 65.25 | 47739.65 | 48.00 | 0.7356 |
| 2 | 4352.41 | 11.67 | 39131.32 | 44.17 | 3.7850 |
| 3 | 17074.67 | 14.64 | 9081.32 | 16.93 | 1.1570 |
| 4 | 8957.72 | 18.42 | 8960.55 | 20.93 | 1.1367 |
| 5 | 3111.70 | 20.52 | 8041.13 | 44.26 | 2.1574 |
| 6 | 10542.12 | 11.00 | 19300.65 | 18.71 | 1.7013 |
| 7 | 2296.42 | 37.65 | 1786.04 | 20.30 | 0.5391 |

As a result, it was found that samples in which the expressed amount of STP3 gene transcript was increased by a factor of 10% or more were frequently found in the canceration-caused stomach tissues. Based on this result, it was indicated that morbid state tissues of the stomach having increased amount of expression in comparison with the ex s amount of the DNA of the present invention in healthy tissues have a high possibility of generating canceration, so that it was indicated that the expressed amount of the DNA of the present invention is useful for the detection of canceration.

### EXAMPLE 4

### Change in the Expressed Amount of the DNA of the Present Invention in Large Bowel Cancer Tissue

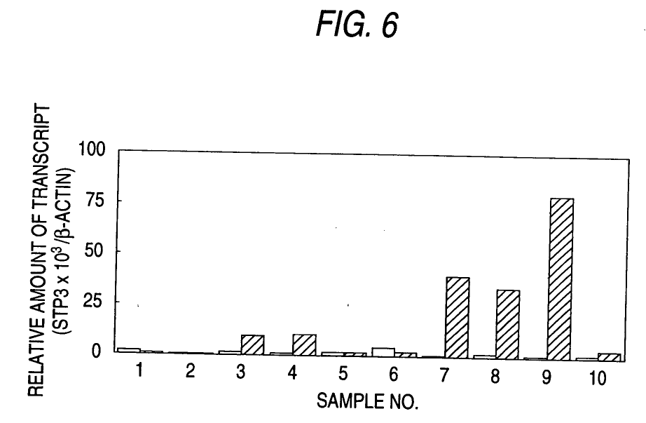

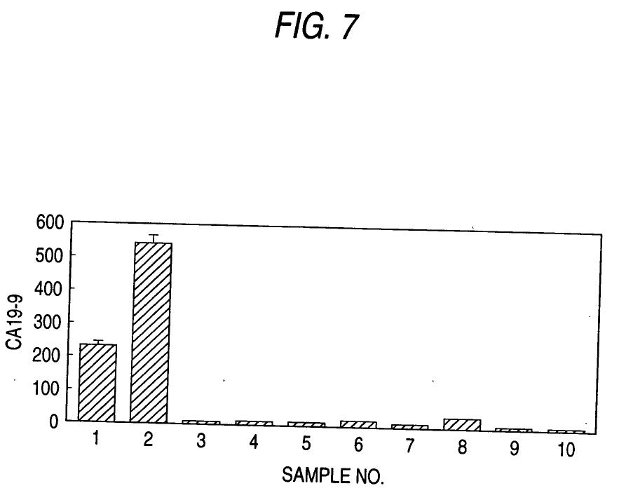

Using a quantitative real time PCR, expressed amount of the DNA of the present invention in a human large intestine cancer tissue was compared with that of a healthy large intestine tissue of the same patient. RNA was extracted from a human large bowel cancer tissue or a healthy large intestine tissue using RNease Mini Kit (manufactured by Qiagen) and converted into a single strand DNA by a oligo(dT) method using Super-Script First-Strand Synthesis System (manufactured by Invitrogen). Using this DNA as the template, a quantitative real time PCR was carried out by ABI PRISM 7700 (manufactured by Applied Biosystems) using primers (5′ primer: SEQ ID NO:11, 3′ primer: SEQ ID NO:12) and a TaqMan probe (SEQ ID NO:13). The PCR was carried out by heating at 95° C. for 10 minutes and then 40 cycles of a reaction at 95° C. for 15 seconds and at 60° C. for 1 minute as one cycle. Using a β-actin gene as an internal standard, comparison was made by calculating ratio of the expressed amount of the STP3 gene transcript to the expressed amount of the β-actin gene (b-Act) transcript (Table 2, FIG. 6). Also, as a reference, CA19-9 in the same samples was measured (Table 2, FIG. 7).

TABLE 2

| Sam- | Healthy tissue | | Canceration tissue | | Canceration | |
|---|---|---|---|---|---|---|
| ple | Measured | STP3/ | Measured | STP3/ | tissue/ | |
| No. | value | b-Act | value | b-Act | healthy | CA19-9 |
| 1 | 383.33 | 1.27 | 283.26 | 0.42 | 0.3305 | 233 |
| 2 | 195.12 | 0.15 | 144.40 | 0.15 | 0.9781 | 540 |
| 3 | <<40.10 | 0.92 | 3021.63 | 9.20 | 9.9705 | 7 |
| 4 | 103.39 | 0.19 | 14875.02 | 9.91 | 51.2905 | 10 |
| 5 | 153.30 | 1.15 | 1314.66 | 1.33 | 1.1606 | 11 |
| 6 | 1901.04 | 3.82 | 1765.64 | 1.71 | 0.4474 | 17 |
| 7 | <<40.10 | 0.04 | 28835.00 | 39.25 | 950.4352 | 9 |
| 8 | 480.98 | 0.95 | 51125.24 | 33.92 | 35.8110 | 30 |
| 9 | 215.04 | 0.31 | 33519.91 | 79.83 | 253.9229 | 7 |
| 10 | 132.05 | 0.26 | 3046.17 | 3.55 | 13.4816 | 6 |

As a result, it was found that samples showing the increased expressed amount of STP3 gene transcript were frequent in the canceration-caused large intestine tissues. Also, since expressed amount of STP3 gene transcript was increased in samples having low measured value of CA19-9, it was shown that the detection method of the present invention can detect canceration-caused tissues which could not be detected with CA19-9.

While the present invention has been described in detail and with reference to specific embodiments thereof, it will be apparent to one of skill in the art that various changes and modifications can be made therein without departing from the spirit and scope thereof. All references cited herein are incorporated in their entirety.

This application is based on Japanese application No. 2002-382123 filed on Dec. 27, 2002, the entire contents of which are incorporated hereinto by reference.

What is claimed is:

1. A protein of the following (a) or (b):
(a) a protein comprising the amino acid sequence represented by SEQ ID NO:2,
(b) a protein comprising an amino acid sequence in which 1 to 21 amino acid residue(s) are substituted, deleted, inserted or transposed in the amino acid sequence represented by SEQ ID NO:2 and also having active sulfate transport activity.

2. The protein according to claim 1, wherein the active sulfate transport activity is 3′-phosphoadenosine-5′-phosphosulfate transport activity.

3. An active sulfate transporter agent which comprises the protein according to claim 1 as an active ingredient.

4. A method for transporting active sulfate, which comprises contacting active sulfate with the protein according to claim 1.

5. A method for detecting canceration of a tissue to be tested, which comprises relating a detected value of expression in a tissue to be tested of the protein according to claim 1 to canceration of the tissue to be tested.

6. The method according to claim 5, wherein the detected value of expression in a tissue to be tested of the protein is a difference obtained by determining an expressed amount of a nucleic acid encoding the protein and comparing the value obtained by the determination with the expressed amount of the nucleic acid in a healthy tissue.

7. The method according to claim 6, wherein the expressed amount of a nucleic acid encoding the protein is the expressed amount of a nucleic acid comprising the nucleotide sequence of the following (A) or (B):
(A) a nucleotide sequence of 30 to 1,500 bp comprising a part of the nucleotide sequence represented by SEQ ID NO:1,
(B) a nucleotide sequence of 30 to 1,500 bp comprising a nucleotide sequence complementary to a part of the nucleotide sequence represented by SEQ ID NO:1.

8. The method according to claim 7, wherein the part of the nucleotide sequence represented by SEQ ID NO:1 is a nucleotide sequence of nucleotide numbers 145 to 1,443 resented by SEQ ID NO:1 or a nucleotide sequence of nucleotide numbers 464 to 553 represented by SEQ ID NO:1.

9. The method according-to claim 5, wherein the tissue to be tested is a tissue derived from the stomach or the large intestine.

10. A nucleic acid of 30 to 1,500 bp which comprises a part of the full nucleotide sequence represented by SEQ ID NO:1, or a nucleotide sequence complementary to the part of the nucleotide sequence.

11. The nucleic acid according to claim 10, which comprises a nucleotide sequence of nucleotide numbers 464 to 553 represented by SEQ ID NO:1, or a nucleotide sequence complementary to the nucleotide sequence.

12. The nucleic acid according to claim 10, which comprises a nucleotide sequence of nucleotide numbers 145 to 1,443 represented by SEQ ID NO:1, or a nucleotide sequence complementary to the nucleotide sequence.

13. The nucleic acid according to claim 10, which consists of a nucleotide sequence of nucleotide number 145 to 1,443 represented by SEQ ID NO:1, or a nucleotide sequence complementary to the nucleotide sequence.

14. The nucleic acid according to claim 10, which is a DNA.
